(19) United States
(12) Patent Application Publication
(10) Pub. No.: US 2010/0241032 A1
(43) Pub. Date: Sep. 23, 2010

(54) METHOD OF STOPPING A BLEEDING FROM A FISTULA USING A TOURNIQUET DEVICE

(76) Inventors: Tsung-Lung Lee, Kaohsiung City (TW); Yu-Li Kang, Tainan City (TW)

Correspondence Address: DICKSTEIN SHAPIRO LLP, 1825 EYE STREET NW, Washington, DC, 20006-5403, US

(21) Appl. No.: 12/710,415

(22) Filed: Feb. 23, 2010

(30) Foreign Application Priority Data
Mar. 19, 2009 (TW) 098204342

(51) Int. Cl. A61B 7/00; A61B 17/00

(52) U.S. Cl. 600/586; 606/203

(57) ABSTRACT

A method of stopping a bleeding from a fistula situated on a limb of a patient for hemodialysis, includes: providing a tourniquet band having a blood-stopping protrusion projecting therefrom; encircling the limb of the patient with the tourniquet band to cover the fistula and pressing the fistula with the blood-stopping protrusion to stop the bleeding; connecting a sensor to the tourniquet band to sense a blood flow in the fistula and to generate a signal representative of the blood flow; and electrically connecting a notifying device to the sensor and using the notifying device to produce a notifying signal in response to the signal transmitted by the sensor.

### CROSS-REFERENCE TO RELATED APPLICATION

This application claims priority of Taiwanese application No. 098204342, filed on Mar. 19, 2009.

### BACKGROUND OF THE INVENTION

1. Field of the Invention

This invention relates to a method of stopping a bleeding, more particularly to a method of stopping a bleeding from a fistula using a tourniquet device.

2. Description of the Related Art

When a dialysis needle is removed from a dialysis fistula, which usually has a high blood pressure, after kidney dialysis treatment, a constrictive pressure is necessary for preventing blood loss from the dialysis fistula. Generally, the time required to stop a bleeding is about two hours.

A conventional method for stopping a bleeding is to use a tourniquet to constrict a patient's arm. However, the dialysis fistula may become narrow due to the constriction such that blood flow is adversely affected to become turbulent and may lead to blood clotting, which endangers the patient. Consequently, during the process of stopping the bleeding, the patient needs to detect the blood flow in the fistula periodically using a stethoscope so as to timely adjust the tourniquet to allow the blood to fluently flow in the fistula. If the patient forgets to detect the blood flow in the fistula, or the patient has a low sensitivity to a sound change or misunderstands the sound change, the patient's health may be in danger. In addition, the method for detecting the blood flow in the fistula using a stethoscope is inconvenient and is not suitable for a deaf patient.

### SUMMARY OF THE INVENTION

Therefore, an object of the present invention is to provide a method of stopping a bleeding from a fistula that can overcome the aforesaid drawbacks associated with the prior art.

According to the present invention, a method of stopping a bleeding from a fistula situated on a limb of a patient for hemodialysis, comprises: providing a tourniquet band having a blood-stopping protrusion projecting therefrom; encircling the limb of the patient with the tourniquet band to cover the fistula and pressing the fistula with the blood-stopping protrusion to stop the bleeding; connecting a sensor to the tourniquet band to sense a blood flow in the fistula and to generate a signal representative of the blood flow; and electrically connecting a notifying device to the sensor and using the notifying device to produce a notifying signal in response to the signal transmitted by the sensor.

### BRIEF DESCRIPTION OF THE DRAWINGS

Other features and advantages of the present invention will become apparent in the following detailed description of the preferred embodiment of this invention, with reference to the accompanying drawings, in which:

FIG. 1 is a perspective view of a tourniquet device used in a preferred embodiment of the method according to this invention;

FIG. 2 is a fragmentary sectional view of the tourniquet device; and

FIG. 3 is a schematic view illustrating the tourniquet device wound on an arm to perform the preferred embodiment of the method according to this invention.

### DETAILED DESCRIPTION OF THE PREFERRED EMBODIMENT

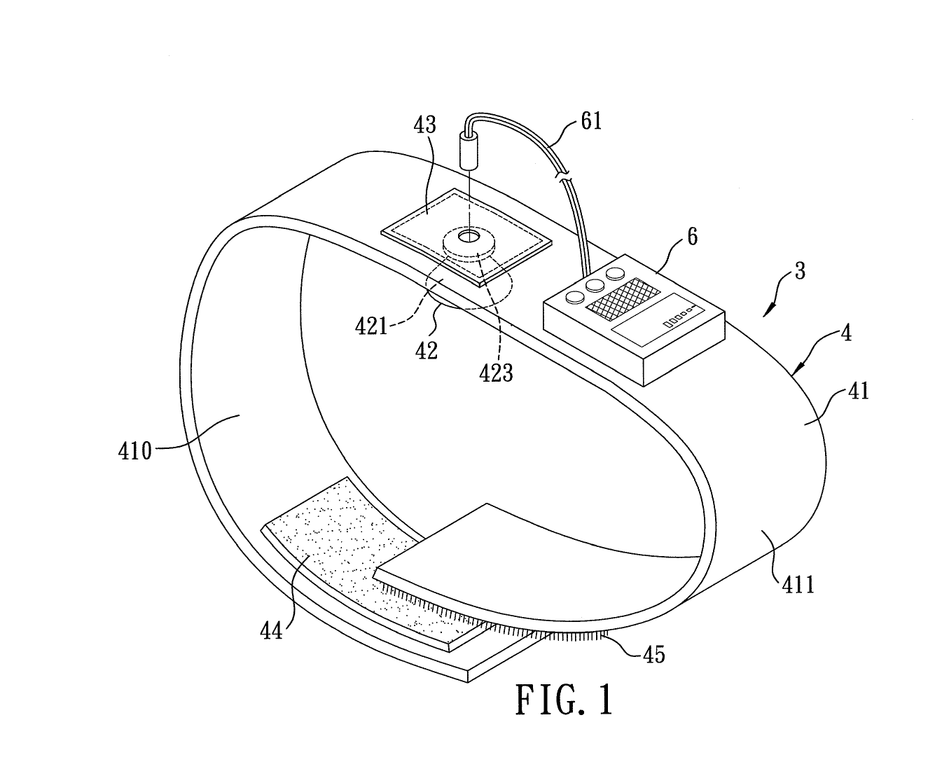

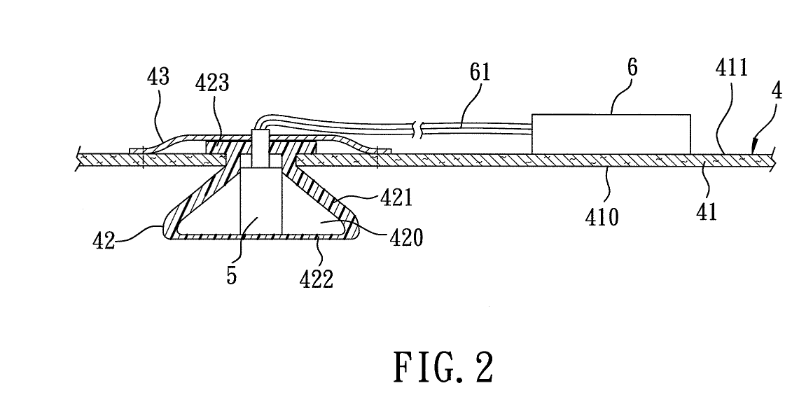

A method of stopping a bleeding from a fistula situated on a limb of a patient for hemodialysis according to the invention is embodied using a tourniquet device 3 as shown in FIGS. 1 and 2. The tourniquet device 3 is adapted to encircle a limb of a patient for stopping the bleeding from a fistula for hemodialysis and for detecting the blood flow in the fistula.

A method according to a preferred embodiment of the invention includes a first step of providing a tourniquet band having a blood-stopping protrusion projecting therefrom. A suitable tourniquet band for the method may be an elongated tourniquet band 4 of the tourniquet device 3, which includes a band body 41 that has an abutment surface 410 and an outer surface 411 opposite to the abutment surface 410, a hollow blood-stopping protrusion 42 projecting inwardly from the band body 41, a fixing member 43 to affix the blood-stopping protrusion 42 to the band body 41, and first and second fasteners 44, 45 which are attached to the band body 41 and which are engaged detachably with each other. In this embodiment, the first and second fasteners 44, 45 are hook-and-loop fasteners which are interlockable with each other.

The hollow blood-stopping protrusion 42 is made from a waterproof material, such as metal or plastic. In this embodiment, the hollow blood-stopping protrusion 42 is made from plastic. The hollow blood-stopping protrusion 42 has a resilient membrane portion 422 adapted to abut against the patient's limb, a substantially conical wall 421 extending convergingly from the resilient membrane portion 422 to the outer surface 411 by penetrating the band body 41, and an enlarged retaining portion 423 connected to the conical wall 421 and abutting against the outer surface 411. The fixing member 43 is used to affix the enlarged retaining portion 423 to the outer surface 411 of the band body 41. The resilient membrane portion 422 cooperates with the conical wall 421 to define a space 420.

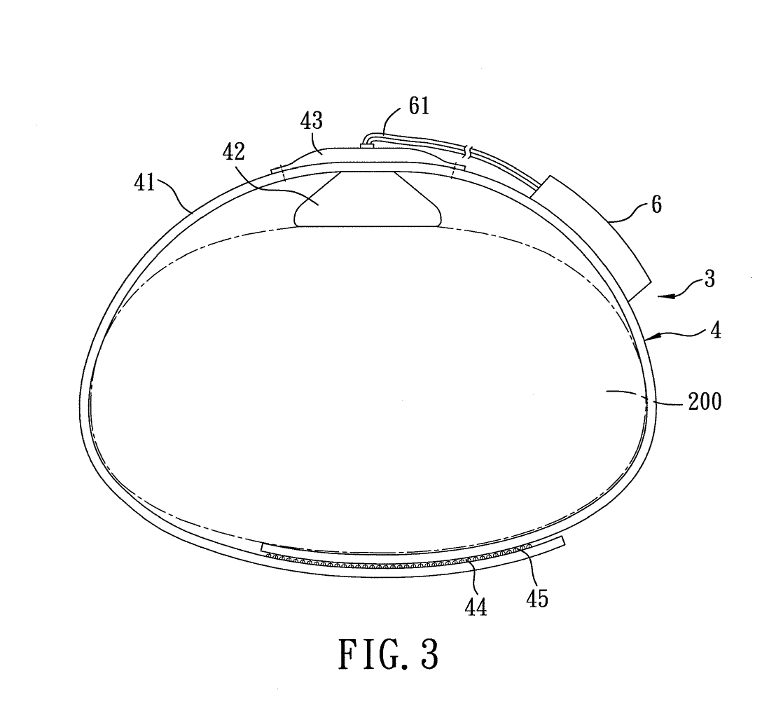

Referring to FIG. 3, the method of the invention further includes the step of encircling a limb or arm 200 of the patient with the tourniquet band 4 to cover the fistula (not shown) and pressing the fistula with the blood-stopping protrusion 42 to stop the bleeding from the fistula. The step is carried out after a dialysis needle is removed from the fistula. The resilient membrane portion 422 of the hollow blood-stopping protrusion 42 is pressed on the fistula while the patient's arm 200 is encircled with the tourniquet band 4 using the first and second fasteners 44, 45 to tighten the tourniquet band 4. By virtue of the first and second fasteners 44, 45, the band body 41 can be tightened or loosened.

The method further includes the steps of: connecting a sensor to the tourniquet band to sense a blood flow in the fistula and to generate a signal representative of the blood flow; and electrically connecting a notifying device to the sensor and using the notifying device to produce a notifying signal in response to the signal transmitted by the sensor.

Referring once again to FIGS. 1 and 2, the sensor and the notifying device suitable for the method are respectively shown at 5 and 6. The sensor 5 may be a sound sensor (e.g. microphone) for detecting a sound of the blood flow in the fistula and generating a sound signal representative of the sound of the blood flow in the fistula. The sensor 5 is connected to the tourniquet band 4 and is disposed inside the space 420 within the blood-stopping protrusion 42.

The notifying device 6 is detachably mounted on the band body 41 and is electrically connected to the sensor 5 through a signal transmission cable 61. The cable 61 has a distal end penetrating through the fixing member 43 and the enlarged retaining portion 423, and electrically inserted into the sensor 5 such that the notifying device 6 can produce a signal in response to the signal transmitted by the sensor 5 for the patient to understand the blood flow condition in the fistula.

In this embodiment, the notifying device 6 is mounted on the band body 41 in a hook-and-loop engaging manner and includes a sound amplifier and an image display device.

The sensor 5 is used to continuously sense and generate the sound signal representative of the blood flow in the fistula that is pressurized, and the notifying device 6 is operated to automatically amplify the sound signal and produce an image signal in response to the sound signal transmitted by the sensor 5 for the patient to determine if the blood flow in the fistula is normal. In addition, by virtue of the notifying device 6, use of a stethoscope can be avoided, and medical personnel can observe and teach the patient to understand what the sound signal represents. For example, when a specific sound is produced, there will be a blood clot in the fistula. It is worth mentioning that when blood flows rapidly through the fistula, a thrill can be felt. Conversely, when the fistula is clogged, the thrill disappears and a throbbing sensation in the fistula in time with the heartbeat is present. Since the throbbing sensation causes deformation of the resilient membrane portion 422 of the hollow blood-stopping protrusion 42 so as to produce a pressure change, the sensor 5 can alternatively be a pressure-measuring sensor that detects the pressure change.

Furthermore, since blood red cells contain hemoglobin and hemoglobin can absorb infrared ray, a concentration of the blood red cells in the fistula can be detected using an infrared sensor unit. Therefore, the sensor 5 can be an infrared sensor. In this case, the resilient membrane portion 422 of the hollow blood-stopping protrusion 42 may be made of a transparent material. The infrared sensor can emit infrared ray toward the fistula through the resilient membrane portion 422 and receives the reflected part of the infrared ray which is not absorbed by the blood red cells. According to the reflected infrared ray, the sensor 5 generates a signal and the notifying device 6 produces a signal in response to the signal transmitted by the sensor 5.

In use, when blood flows fluently through the fistula, the infrared ray emitting from the infrared sensor is absorbed by the blood red cells in the fistula, so that the infrared sensor will detect a weak reflected infrared ray. On the contrary, when the fistula is clogged, less blood flows through the fistula such that absorption of the infrared ray is reduced and a strong reflected infrared ray will be detected.

In this embodiment, the notifying device 6 includes the sound amplifier and the image display device, which is not suitable for disabled patients having problems of sense of hearing, and sight. The notifying device 6 can be a gas release device for producing a flavor signal representative of the blood flow in the fistula, or a deformable device for producing a shape signal representative of the blood flow in the fistula, or a liquid release device for producing a taste signal representative of the blood flow in the fistula.

Moreover, when the band body 41 or the sensor 5 is damaged, the notifying device 6 can be reused by separating from the tourniquet band 4 for connecting to a new band body and a sensor. The type of the notifying device 6 can be varied depending on the actual requirement.

In addition, the sensor 5 can be mounted on the band body 41 and is directly pressed on the patient's arm 200 for sensing a blood flow in the fistula in other embodiments of the invention.

By virtue of the method of the invention, a bleeding from the fistula can be stopped, and the patient can understand the blood flow in the fistula through the notifying device 6.

With the invention thus explained, it is apparent that various modifications and variations can be made without departing from the spirit of the present invention. It is therefore intended that the invention be limited only as recited in the appended claims.

What is claimed is:

1. A method of stopping a bleeding from a fistula situated on a limb of a patient for hemodialysis, comprising:
providing a tourniquet band having a blood-stopping protrusion projecting therefrom;
encircling the limb of the patient with the tourniquet band to cover the fistula and pressing the fistula with the blood-stopping protrusion to stop the bleeding;
connecting a sensor to the tourniquet band to sense a blood flow in the fistula and to generate a signal representative of the blood flow; and
electrically connecting a notifying device to the sensor and using the notifying device to produce a notifying signal in response to the signal transmitted by the sensor.

2. The method of claim 1, wherein the sensor is a sound sensor that detects a sound of the blood flow in the fistula.

3. The method of claim 1, wherein the sensor is disposed within the blood-stopping protrusion.

4. The method of claim 1, wherein the sensor is a pressure-measuring sensor that detects a pressure change caused by the blood flow in the fistula.

5. The method of claim 1, wherein the sensor is an infrared ray sensor that detects a concentration of blood red cells in the fistula.

6. The method of claim 1, wherein the notifying device is an image display device for producing an image signal representative of the blood flow in the fistula.

7. The method of claim 1, wherein the notifying device is a sound amplifier for producing a sound signal representative of the blood flow in the fistula.

8. The method of claim 1, wherein the notifying device is a deformable device for producing a shape signal representative of the blood flow in the fistula.

9. The tourniquet device of claim 1, wherein the notifying device is a gas release device for producing a flavor signal representative of the blood flow in the fistula.

10. The method of claim 1, wherein the notifying device is a liquid release device for producing a taste signal representative of the blood flow in the fistula.

11. The method of claim 1, wherein the notifying device is able to produce image and sound signals representative of the blood flow in the fistula.
